(19) United States
(12) Patent Application Publication
(10) Pub. No.: US 2009/0192528 A1
(43) Pub. Date: Jul. 30, 2009

(54) METHOD AND DEVICE FOR HERNIA REPAIR

(75) Inventors: Joel C. Higgins, Claypool, IN (US); Matthew Swift, Fort Wayne, IN (US)

Correspondence Address: HARNESS, DICKEY & PIERCE, P.L.C., P.O. BOX 828, BLOOMFIELD HILLS, MI, 48303, US

(73) Assignee: Biomet Biologics, Inc., Warsaw, IN (US)

(21) Appl. No.: 12/021,739

(22) Filed: Jan. 29, 2008

(51) Int. Cl. A61B 17/11; A61F 2/02

(52) U.S. Cl. 606/151; 623/23.72

(57) ABSTRACT

A device for repairing a hernia defect in a patient, the device having a biocompatible mesh material having a treated surface and a tissue composition coated on at least a portion of the treated surface, where the tissue composition originates from the patient. A method of preparing a surgical mesh for repairing a hernia defect, comprises placing a tissue specimen into a container, centrifuging the container to separate the specimen into at least two fractions, then drawing a selected fraction from the container, treating a surface of a biocompatible mesh and coating the surface-treated biocompatible mesh with the selected fraction.

The present technology relates to methods and devices used in repairing hernias and to promote or enhance tissue repair related thereto.

In many hernia repairs, a small piece of mesh is used to repair a weakness in tissue. For example, an incision is made at the site of the hernia and a piece of mesh is inserted to cover the area of an abdominal wall defect without sewing together the surrounding tissue. The mesh is biocompatible and generally well-accepted by the body's natural tissues.

Nevertheless, although mesh products have revolutionized hernia repair, in some cases there may be mesh-related complications, such as incomplete healing. (See, for example, Robinson et al., Surg Endosc (2005) 19:1556-1560). There is a need for new methods of preparing and using meshes that are used in hernia repair.

### SUMMARY

The present technology provides devices for repairing a hernia defect in a subject, the devices comprising a biocompatible mesh material having a treated surface and an isolated tissue composition coated on at least a portion of the treated surface. In some devices, the isolated tissue composition originates from the subject.

The present technology also provides methods of preparing a surgical mesh for repairing a hernia defect. Such methods include placing a tissue specimen into a container, centrifuging the container to separate the specimen into at least three fractions, drawing a selected fraction from the container, and coating a surface of a surface-treated biocompatible mesh with the selected fraction.

Further areas of applicability will become apparent from the description provided herein. It should be understood that the description and specific examples are intended for purposes of illustration only and are not intended to limit the scope of the present teachings.

### DRAWINGS

The present technology will become more fully understood from the detailed description and the accompanying drawings, wherein:

FIG. 1 illustrates a representative site of a hernia defect on a subject in need of treatment according to some embodiments of the present technology;

FIG. 2 is a diagrammatic illustration of a representative method for treating a hernia defect according to one embodiment of the present technology;

FIG. 3 is a cross-sectional view of a representative device used for isolating a blood component according to one embodiment of the present technology;

FIGS. 4A and 4B are cross-sectional views of a representative device used for forming a concentrated blood component according to one embodiment of the present technology; and

FIG. 5 illustrates a representative manner of administering a hernia defect treatment to the subject according to one embodiment of the present technology.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom.

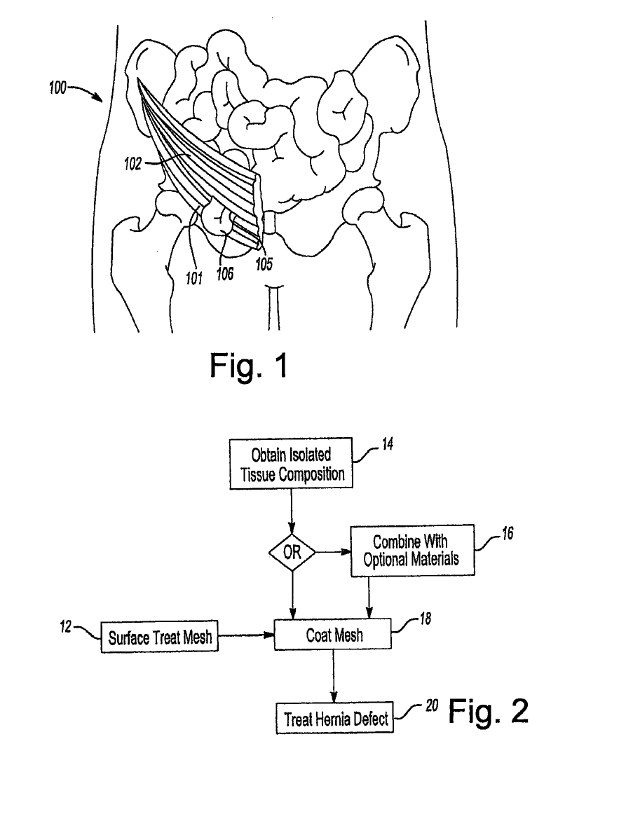

Referring to FIG. 1, a hernia defect 101 in a subject 100 is the formation of an opening 105 in the abdominal wall 102. In many cases, internal tissue, for example, an intestine 106, protrudes through the opening 105. Treatment for a hernia defect typically involves surgery to replace the protruding internal organ 106 or abdominal tissue inside the abdominal wall 102, followed by repair of the opening 105.

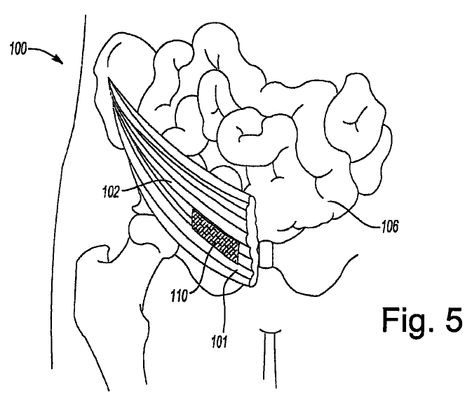

One general method for treating a hernia defect 101 is shown diagrammatically in FIG. 2. In summary, an isolated tissue composition is obtained at step 14. Optional materials may also be combined with the isolated tissue composition at step 16. A mesh suitable for repairing the hernia defect 101 is surface-treated at step 12. The isolated tissue composition formed at step 14, with optional materials added at step 16, is then coated on to at least a portion of the surface-treated mesh at step 18 to create the implant. As further shown in FIG. 5, the implant 110 is then implanted at the site of the hernia defect 101. Each of these steps will be more fully discussed below.

In particular, an isolated tissue composition, such as a blood component, is initially obtained at step 14. The tissue composition may comprise autologous tissue material derived from a tissue specimen from the subject 100 exhibiting the hernia defect 101, or tissue material from another human or animal donor identified as being compatible with the subject 100. The isolated tissue composition may comprise the tissue specimen without further processing, or tissue material that has been isolated or otherwise processed from the tissue specimen. Examples of isolated tissue compositions include platelet-rich plasma, platelet-poor plasma, concentrated platelet-poor plasma, cryoprecipitated plasma, bone marrow aspirate, concentrated bone marrow aspirate, and processed lipoaspirate cells. Some isolated tissue compositions comprise at least one of hematopoietic stem cells, stromal stem cells, mesenchymal stem cells, endothelial progenitor cells, red blood cells, white blood cells, fibroblasts, reticulocytes, adipose cells, thrombocytes and endothelial cells.

An isolated tissue composition comprising platelet-rich plasma may have an increased concentration of platelets relative to whole blood. For example, the platelet concentration can be from about 3-fold to about 10-fold greater than the platelet concentration in whole blood. Alternatively, an isolated tissue composition comprising platelet-poor plasma may have a decreased concentration of platelets relative to whole blood, such as from about 0 to about 100,000 platelets/mL. The platelet-poor plasma can also be concentrated to make concentrated platelet-poor plasma. Further, the isolated tissue composition obtained at step 14 may comprise combinations of fractionated plasma. For example, the isolated tissue composition may have varying proportions of platelet-rich plasma and platelet-poor plasma, resulting in ranges of platelet concentrations that are continuous from platelet-rich plasma to platelet-poor plasma. The isolated tissue composition may also comprise platelet-rich plasma or isolated platelets, either of which may be diluted and/or resuspended with platelet-poor plasma or concentrated platelet-poor plasma.

An isolated tissue composition can be obtained at step 14 by one or more methods, including filtration, cryoprecipitation, and density fractionation. Density fractionation techniques include single stage centrifugation, centrifugation in multiple stages, and continuous flow centrifugation.

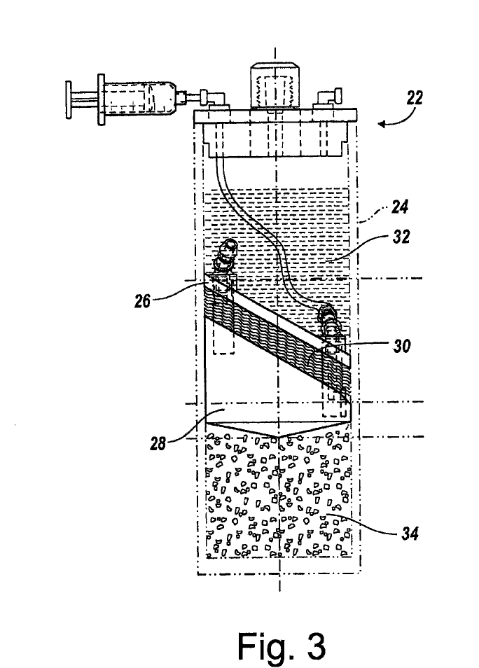

One example of a device that may be used for forming the isolated tissue composition at step 14 is shown in FIG. 3. In this regard, the device 22 includes a container 24, such as a tube, that is placed in a centrifuge after being filled with blood. The container 24 includes a buoy system having an isolator 26 and a buoy 28. The buoy 28 has a selected density which is tuned to reach a selected equilibrium position upon centrifugation; this position lies between a more dense blood fraction and a less dense blood fraction. During centrifugation, the buoy 28 separates the blood within the container 24 into at least two fractions, without substantially commingling the fractions, by sedimenting to a position between the two fractions. In this regard, the isolator 26 and the buoy 28 define a layer comprising platelet-rich plasma 30, while less dense platelet-poor plasma 32 generally fractionates above the isolator 26, and more dense red blood cells 34 generally fractionate below the buoy 28. Following centrifugation, a syringe or tube may then be interconnected with a portion of the buoy system to extract one or more selected fractions for use as the isolated tissue composition. Devices including those disclosed in FIG. 3 and associated methods are described in U.S. Pat. No. 7,179,391, Leach et al., issued Feb. 20, 2007; and U.S. Patent Application Publication 2005/0109716, Leach et al., published May 26, 2005; both of which are incorporated by reference herein. One such device that is commercially available is the GPS® Platelet Concentrate System, from Biomet Biologics, Inc. (Warsaw, Ind.).

Another example of a device that may be used in step 14 to isolate platelet-rich plasma by density frationation comprises a centrifugal drum separator and an erythrocyte capture trap. In one embodiment, the walls of the centrifugal drum separator are coated with a depth filter having pores and passageways that are sized to receive and entrap erythrocytes. Blood is placed in the centrifugal drum, and the drum is spun along its axis at sufficient speed so as to force erythrocytes from the blood into the depth filter. After spinning, the erythrocytes remain in the filter and the remaining platelet-rich plasma is extracted. The platelet-rich plasma may be concentrated by desiccation. Such devices include the Vortech™ Concentration System (Biomet Biologics, Inc., Warsaw, Ind.), and are disclosed in U.S. Patent Application Publication 2006/0175244, Dorian et al., published Aug. 10, 2006 and U.S. Patent Application Publication 2006/0175242, Dorian et al., published Aug. 10, 2006, which are hereby incorporated by reference. Such devices may be used to prepare platelet-rich plasma in lieu of or in addition to using the tube having a buoy that is described above and shown in FIG. 3.

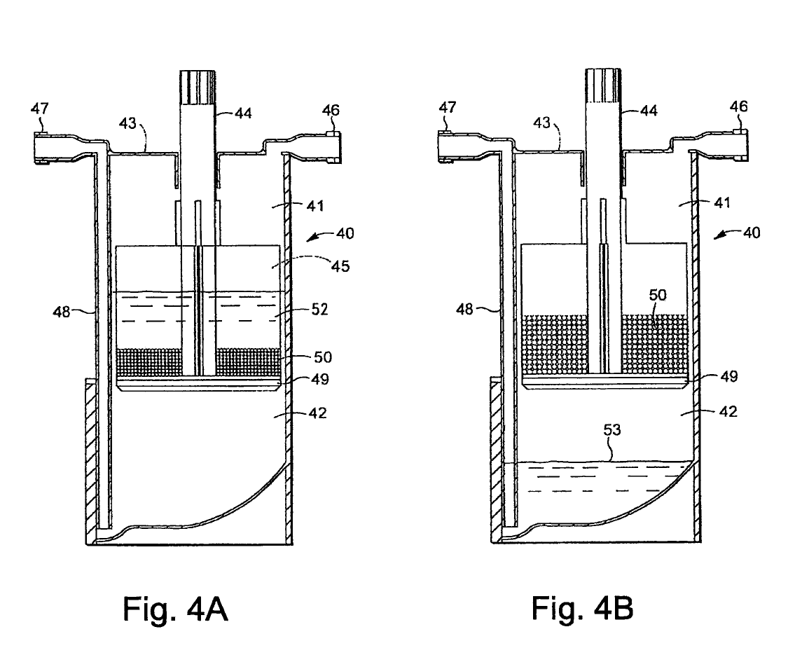

The isolated tissue composition obtained at step 14 may contain concentrated platelet-poor plasma. One example of a device that may be used for forming concentrated platelet-poor plasma at step 14 is shown in FIGS. 4A and 4B. In this regard, the device 40 has an upper chamber 41 and a lower chamber 42. The upper chamber 41 has an end wall 43 through which the agitator stem 44 of a gel bead agitator 45 extends. The device 40 also has a plasma inlet port 46 that extends through the end wall 43 and into the upper chamber 41. The device 40 also includes a plasma concentrate outlet port 47 that communicates with a plasma concentrate conduit 48. The floor of upper chamber 41 includes a filter 49, the upper surface of which supports desiccated concentrating polyacrylamide beads 50.

During use, blood plasma 52 (preferably cell free) is initially introduced into the upper chamber 41 through the plasma inlet port 46. The plasma 52 entering the upper chamber 41 flows to the bottom of the chamber where it contacts the polyacrylamide beads 50 as shown in FIG. 4A. As the polyacrylamide beads 50 remove water from plasma 52, the plasma proteins are concentrated. During this concentration stage, the plasma and its components can be concentrated to a concentration from about 1.5 to about 3 times or higher than its original concentration.

Referring to FIG. 4B, the device 40 is then placed in the cup receptors of a conventional laboratory centrifuge (not shown) and spun at a speed that will create a centrifugal force that will remove plasma concentrate 53 from the polyacrylamide beads 50, and cause the plasma concentrate 53 to flow through the filter 49. The filter 49 can be constructed to allow flow of liquid there-through at centrifugal forces above 10 g. After centrifugation is completed, the device 40 is removed from the centrifuge. The plasma concentrate 53 is then drawn from lower chamber 42 through conduit 48 to the plasma concentrate outlet 47.

Exemplary plasma concentration devices are disclosed in U.S. Patent Application Publication 2006/0175268, Dorian et al., published Aug. 10, 2006; and U.S. Patent Application Publication 2006/0243676, Swift et al., published Nov. 2, 2006; both of which are incorporated by reference herein. Such a device is commercially available as Plasmax™ Plus Plasma Concentrator, from Biomet Biologics, Inc. (Warsaw, Ind.).

Other devices that may be used to obtain the isolated tissue composition at step 14 are described, for example, in U.S. Pat. No. 6,398,972, Blasetti et al., issued Jun. 4, 2002; U.S. Pat. No. 6,649,072, Brandt et al., issued Nov. 18, 2003; U.S. Pat. No. 6,790,371, Dolecek, issued Sep. 14, 2004; and U.S. Pat. No. 7,011,852, Sukavaneshvar et al., issued Mar. 14, 2006; and U.S. Pat. No. 7,223,346, Dorian et al., issued May 29, 2007. In addition to the GPS® Platelet Concentrate System and Vortech™ Concentration System, other commercially available devices may be used to obtain the isolated tissue composition at step 14 include the Megellan™ Autologous Platelet Separator System, commercially available from Medtronic, Inc. (Minneapolis, Minn.); SmartPReP™, commercially available from Harvest Technologies Corporation (Plymouth, Mass.); DePuy (Warsaw, Ind.); the AutoloGel™ Process, commercially available from Cytomedix (Rockville, Md.), and the GenesisCS component concentrating system, available from EmCyte Corporation (Fort Myers, Fla.).

In some embodiments, the isolated tissue composition obtained in step 14 comprises bone marrow aspirate or concentrated bone marrow aspirate. Bone marrow aspirate may be obtained in any appropriate manner, such as from the intramedullary area of a bone by use of a syringe and needle. The bone marrow aspirate may be used as-is in step 14, or may be further processed to create bone marrow concentrate or other isolated tissue composition. A concentrated bone marrow aspirate may be obtained comprising nucleated cells such as red and white blood cells, bone marrow stromal cells, and mesenchymal stem cells.

In some embodiments, a density fractionation device such as shown in FIG. 3 may be used to process the bone marrow aspirate. The bone marrow aspirate can be concentrated by itself, or in combination with whole blood. For example, a mixture of whole blood and bone marrow aspirate may be added to the device shown in FIG. 3, and a buffy coat fraction obtained that contains at least a 4 times greater concentration of nucleated cells from bone marrow. Methods of obtaining an isolated tissue composition from bone marrow aspirate are disclosed in U.S. Patent Application Publication No. 2006/0278588, Woodell-May, published Dec. 14, 2006, incorporated by reference herein.

The isolated tissue composition of step 14 may comprise stem cells, such as bone marrow-derived stem cells and adipose-derived stromal cells. In one method, adipose-derived stromal cells may be obtained from processing of lipid tissue by standard liposuction and lipoaspiration methods known in the art. Adipose tissue may also be treated with digestive enzymes and with chelating agents that weaken the connections between neighboring cells, making it possible to disperse the tissue into a suspension of individual cells without appreciable cell breakage. In another method, bone marrow derived stromal cells may be isolated from bone marrow tissue, including bone marrow aspirate harvested by methods known in the art. Methods may also include compositions of stromal cells comprising both adipose stromal cells and bone marrow derived stromal cells. Stromal cells from adipose and bone marrow tissues may be obtained from the same organism or from different organisms.

Following disaggregation, the adipose stromal cells may be isolated from the suspension of cells and disaggregated tissue, such as adipose tissue or bone marrow aspirate. A device as shown in FIG. 3, such as the GPS® Platelet Concentrate System, may be used to isolate adipose stromal cells.

In some embodiments, the isolated tissue composition obtained in step 14 is combined with one or more optional materials in step 16. Such optional materials include, for example, platelet activators, albumin binding agents, scaffolds, bioactive materials, cytokines, and combinations thereof. The optional materials can be applied in step 16 just prior to the administration of the isolated tissue composition in step 18. Alternatively, the optional materials may be applied in step 16 concomitant with administration of the isolated tissue composition in step 18, or following administration of the isolated tissue composition to the mesh in step 18.

Platelet activators may be added in step 16 so as to activate one or more growth factors within platelets contained in an isolated tissue composition. Activation of platelets by platelet activators can be performed just prior to administration of the isolated tissue composition, concomitant with administration of the isolated tissue composition, or following administration of the isolated tissue composition to the mesh. Platelet activators among those useful herein include thrombin (such as autologous thrombin), calcium salts (e.g., calcium chloride), collagen, coagulation factors, and mixtures thereof. Coagulation factors include one or more of Factors: V, VII, VIIa, IX, IXaβ, X, Xa, XI, XIa, XII, α-XIIa, β-XIIa, and XIII.

Albumin binding agents may be added in step 16. In various embodiments, an albumin binding agent is combined with plasma. Albumin binding agents include those known in the art, such as polyethylene glycol and bilirubin.

In some embodiments, step 16 may also include the addition of one or more bioactive materials that provide a therapeutic, nutritional or cosmetic benefit to the subject in which implants are applied. Such benefits may include repairing unhealthy or damaged tissue, minimizing infection at the site of implant 110, increasing integration of healthy tissue into the implant 110, and preventing disease or defects in healthy or damaged tissue.

Bioactive materials that may be included in step 16 include organic molecules, proteins, peptides, peptidomimetics, nucleic acids, nucleoproteins, antisense molecules, polysaccharides, glycoproteins, lipoproteins, carbohydrates, and polysaccharides; synthetic and biologically engineered analogs thereof; living cells such as chondrocytes, bone marrow cells, stem cells, viruses and virus particles, natural extracts, and stromal cells; and combinations thereof. Specific non-limiting examples of bioactive materials include cytokines, hormones, antibiotics and other anti-infective agents, hematopoietics, thrombopoietics agents, antiviral agents, antitumoral agents (chemotherapeutic agents), antipyretics, analgesics, anti-inflammatory agents, enzymes, vaccines, immunological agents and adjuvants, cytokines, growth factors, cellular attractants and attachment agents, gene regulators, vitamins, minerals and other nutritionals, platelet activators, and combinations thereof. Bioactive agents may be included that have effects at sites not proximate to the site of the cartilage defect 10, such as (in addition to agents listed above) hematopoietics, thrombopoietics, antidementia agents, antiallergic agents, antidepressants, psychotropic agents, anti-parkinsonian agents, therapeutic agents for osteoporosis, cardiotonics, antiarrythmic agents, vasodilators, antihypertensive agents, diuretics, anti-cholinergic, antidiabetic agents, cholesterol lowering agents, gastrointestinal agents, muscle relaxants, and combinations thereof.

In some embodiments, step 16 may also include the addition of one or more cytokines, including isolated, synthetic or recombinant molecules. Cytokines useful herein include growth factors such as transforming growth factor (TGF-beta), bone morphogenic proteins (BMP, BMP-2, BMP-4, BMP-6, and BMP-7), neurotrophins (NGF, BDNF, and NT3), fibroblast growth factor (FGF), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), vular endothelial growth factor (VEGF), epidermal growth factor (EGF), insulin-like growth factors (IGF-I, IFG-II), and combinations thereof.

Referring again to FIG. 2, the isolated tissue composition is coated onto a mesh that is suitable for treating a hernia defect 101. The mesh preferably has strength sufficient to ensure that the mesh does not break or tear after insertion into a subject 100, and may comprise two or more layers of material. The mesh may have a pore size that allows tissue to penetrate through the mesh after the mesh has been inserted into the subject 100. In some embodiments, the mesh has a “memory,” i.e., the ability to resume its shape after deformation, thereby aiding insertion of the mesh into the subject 100 during surgical operation.

The mesh may be made from any biocompatible synthetic, semi-synthetic material, including plastics and other polymers. In some embodiments, the mesh may be made from absorbable or non-absorbable materials. Materials useful for making meshes include poly(ethylene); polyesters; poly(propylene), poly(propylene) polyesters such as poly(propylene) fumarate; polystyrene; polytetrafluoroethylene (PTFE); nylon; polypropylene/PTFE; polypropylene/cellulose; polypropylene/monochryal; polyester/collagen; poly(acrylate); poly(methyl methacrylate); poly(hydroxyethyl methacrylate); poly(vinyl alcohol); poly(carbonate); poly(trimethylene carbonate); poly(ethylene-co-vinyl acetate); poly(ether urethane); poly(ester urethane); poly(arylate); poly(imide); poly(anhydride-co-imide); poly(amino acid); polydepsipeptide; poly(phospbazene); poly(glycolic acid); poly(lactic acid); poly(lactide-co-glycolide); poly(ε-caprolactone); poly(p-dioxanone); poly(lactide-co-glycolide); poly(ε-caprolactone-co-glycolide); poly(glycolide-co-trimethylene carbonate); lactide/tetramethylglycolide copolymer; lactide/trimethylene carbonate copolymer; lactide-δ-valerolactone copolymer; lactide (ε-captrolactone copolymer); poly(lactide)/polyethylene oxide copolymer; unsymmetrically 3,6-substituted poly(1,4-dioxane-2,5-dione); poly(β-alkanoic acids) such as poly(β-hydroxybutyrate), poly(β-hydroxybutyrate)/(β-hydroxyvalerate) copolymer, poly(β-maleic acid) and poly(β-hydroxypropionate), poly(δ-valerolatone); methylmethacrylate-N-vinyl pyrrolidone copolymer, polyesteramide; polyesters of oxalic acid; polydihydropyran; polyalkyl-2-cyanoacrylate; composites thereof; cellulosic materials, and combinations thereof. Commercially available meshes among those useful herein include Prolene® marketed by Ethicon (Sommerville, N.J.), Sepramesh® marketed by Genzyme Biosurgery (Cambridge, Mass.), and Mycromesh® marketed by WL Gore & Associates (Newark, Del.).

Still referring to FIG. 2, the mesh is surface-treated in step 12 to enhance surface properties such as aiding with cellular attachment, wetting of a polymer surface, and enhancing attachment of tissue compositions. For example, the surface of the mesh may be treated to enhance the surface so that the isolated tissue composition will stick to the surface of the mesh to help facilitate ingrowth of tissue, such as muscle, into the pores of the mesh after implantation. The surface treatment may be of substantially the entire surface of the mesh, or a portion thereof.

Methods of surface treatment in step 12 include plasma treatments such as CASING (cross-linking by activated species of inert gas), plasma etching, plasma deposition, and plasma cleaning; corona discharge; chemical modifications including chemical attack with acidic liquids such as chromic acid; modification by exposure to gamma irradiation in the presence of a reactive gas, such as an acetylene or methane; and combinations thereof. Surface treatments of step 12 may alter the surface region by one or more of: removing a boundary layer, changing surface topography or other physical structure of the surface, changing the chemical nature of the surface; and modifying the hydophilicity or lipophilicity of the surface.

In step 18, the tissue composition is coated on a surface of the surface-treated mesh to create an implant 110. The coating may be on substantially the entire surface of the mesh, or on one or more portions thereof. The coating may be continuous, or may have discontinuities on the surface(s) coated. Coating may be accomplished by any method appropriate for the surface-treated mesh, the composition and the intended use, including by using a syringe, dipping, soaking, swabbing, spraying, or vacuum assisted hydration. The coating may be applied to the surface-treated mesh to create the implant 110 before implanting the mesh onto hernia defect 101, after implanting the mesh, or both.

Still referring to FIG. 2, the implant 110 created in step 18 is implanted at the site of the hernia defect 101 in step 20, according to any medically appropriate procedure. For example, as further illustrated in FIG. 5, the implant 110 can be used to repair an opening 105 in an abdominal wall 102 of subject 100. An incision is made at the site of the hernia defect 101 and the implant 110 is inserted to cover the opening 105 of an abdominal wall 102 without sewing together the surrounding tissues. Alternatively, the implant 110 may be implanted laparoscopically. For example, a laparoscopic surgical technique employs cannulas that extend through a narrow puncture in the abdominal wall 102. Because the abdominal cavity remains closed, the surgeon employs an illuminating optical instrument through one of the cannulas to visualize the hernia defect 101 on a television monitor. Surgical instruments are manipulated by the surgeon through the other cannula in the abdominal wall 102 to place the implant 110 over the abdominal wall opening 105.

The present technology also provides a hernia repair system comprising a consumable component of a density fractionation device, such as the device 22 illustrated in FIG. 3, and a surgical process component. For example, a system may comprise, by reference to FIG. 3, a container 24 comprising includes a buoy system having an isolator 26 and a buoy 28 operable during centrifugation of the container 24 to separate a multi-component tissue specimen into two or more fractions 30, 32, 34 having different densities; and a surgical process component operable to facilitate the repair of a hernial defect 101 in a human or other animal subject 100 using at least one of the fractions. The surgical process component may comprise a surface-treated mesh, operable for coating with one of the fractions 30, 32, 34. The surgical process component may also comprise surgical method instructions for the repair of a hernial defect 101, the instructions comprising steps of obtaining an isolated tissue composition comprising one of the fractions 30, 32, 34, applying the isolated tissue composition to a surface of a surface-treated mesh, and treating the hernial defect 101 with the coated mesh implant 110.

The present technology also provides kits to facilitate the methods described herein. Such kits may comprise one or more components or materials used in making an implant 110 or otherwise used in a method of the present technology. For example, a kit may include one or more of: a surfaced treated mesh; an untreated mesh; a surface treatment composition or device for surface treating a mesh; a syringe or other device for obtaining a tissue specimen from the subject 100; a separator device 22 of FIG. 3 or component thereof for producing a tissue composition from a tissue specimen; a device for applying an isolated tissue composition, such as a blood component, to a mesh; and an anticoagulant or other optional material as described herein.

The kit may include a means of communicating information and/or instructions. A communicating means can illustratively take the form of a label or package insert. In some embodiments, the communication means includes language as required by an organization or government agency such as, for example, the United States Food & Drug Administration. In some embodiments, the communication means can comprise a brochure, advertisement, document, computer readable digital optical reading, such as a diskette or CD, an audio presentation, for example, an audio tape or CD, official presentation, for example, a videotape or DVD, and/or one or more pages on a website.

The embodiments and the examples described herein are exemplary and not intended to be limiting in describing the full scope of the devices, compositions and methods of the present technology. Equivalent changes, modifications and variations can be made within the scope of the present technology, with substantially similar results.

What is claimed is:

1. A device for repairing a hernia defect in a human or other animal subject, comprising:
a biocompatible mesh material having a treated surface; and
an isolated tissue composition coated on at least a portion of said treated surface, wherein said tissue is autologous with said subject.

2. The device according to claim 1 wherein said tissue composition comprises a tissue selected from the group consisting of platelet-rich plasma, platelet-poor plasma, cryoprecipitated plasma, concentrated platelet-poor plasma, fibrin sealant, bone marrow aspirate, concentrated bone marrow aspirate, processed lipoaspirate cells, and combinations thereof.

3. The device according to claim 1 wherein said tissue composition is derived by centrifuging a tissue specimen obtained from said subject.

4. The device according to claim 3 wherein said tissue specimen is selected from the group consisting of whole blood, bone marrow aspirate, lipoaspirate, or combinations thereof.

5. The device according to claim 1 wherein said mesh comprises a polymer selected from the group consisting of poly(glycolide), poly(lactide), poly(ε-caprolactone), poly(trimethylene carbonate), poly(p-dioxanone), poly(lactide-co-glycolide), poly(ε-caprolactone-co-glycolide), poly(glycolide-co-trimethylene carbonate), lactide/tetramethylglycolide copolymer, lactide/trimethylene carbonate copolymer, lactide-δ-valerolactone copolymer, lactide (ε-captolactone) copolymer, polydepsipeptide, poly(lactide)/polyethylene oxide copolymer, unsymmetrically 3,6-substituted poly(1,4-dioxane-2,5-dione), poly(β-hydroxybutyrate), poly(β-hydroxybutyrate)/(β-hydroxyvalerate) copolymer, poly(β-hydroxypropionate), poly(δ-valerolatone), methylmethacrylate-N-vinyl pyrrolidone copolymer, polyesteramide, polyester of oxalic acid, polydihydropyran, polyalkyl-2-cyanoacrylate, polyurethane, poly(vinyl alcohol), polypeptide, poly(β-maleic acid), poly(β-alkanoic acid), poly(propylene) fumarate, cellulosic materials, composites thereof, and combinations thereof.

6. The device according to claim 1 wherein said biocompatible mesh material comprises a compound selected from the group consisting of polypropylene, polyester, polystyrene, polycarbonate, PTFE, polypropylene/PTFE, polypropylene/cellulose, polyester/collagen, nylon, composites thereof, and combinations thereof.

7. The device according to claim 1 wherein said treated surface is formed by plasma etching, plasma cleaning, plasma deposition, corona discharge, chemical attack with acidic liquids, modification by exposure to gamma irradiation in the presence of a reactive gas, or a combination thereof.

8. The device according to claim 1 further comprising an optional material selected from the group consisting of antibiotics, chemotherapeutics, gene therapy agents, anti-inflammatories, clotting agents, antioxidants, growth factors, cytokines and combinations thereof.

9. The device according to claim 1 further comprising at least one exogenous activator of a coagulation cascade, coated on said treated surface.

10. The device according to claim 1 wherein said tissue composition comprises hematopoietic stem cells, stromal stem cells, mesenchymal stem cells, endothelial progenitor cells, microvascular endothelial cells, red blood cells, white blood cells, fibroblasts, reticulocytes, adipose cells, endothelial cells, or combinations thereof.

11. The device according to claim 1, wherein said treated surface comprises substantially the entire surface of said mesh material.

12. A method of preparing a surgical mesh for a repair of a hernia defect in a human or other animal subject, the method comprising:
drawing a first specimen comprising blood from said subject;
drawing a second specimen comprising bone marrow aspirate from said subject;
combining said first specimen and said second specimen into a container operable to fractionate a combination of said first specimen and said second specimen into different densities;
centrifuging said container to separate said combination into at least two fractions;
drawing at least one fraction from the container;
treating a surface of a surgical mesh to form a surface-treated mesh; and
coating said surface with said fraction.

13. The method according to claim 12 wherein the at least one fraction is selected from the group consisting of platelet-rich plasma, platelet-poor plasma, cryoprecipitated plasma, concentrated platelet-poor plasma, fibrin sealant, bone marrow aspirate, concentrated bone marrow aspirate, processed lipoaspirate cells, and combinations thereof.

14. The method according to claim 12 wherein said treating the surface of a surgical mesh is selected from the group consisting essentially of plasma etching, plasma deposition, plasma cleaning, corona discharge, chemical attack with acidic liquids, modification by exposure to gamma irradiation in the presence of a reactive gas, and combinations thereof.

15. The method according to claim 12 further comprising coating said treated surface with an optional material selected from the group consisting of antibiotics, chemotherapeutics, gene therapy agents, anti-inflammatories, clotting agents, antioxidants, growth factors, cytokines or combinations thereof.

16. The method according to claim 12 further comprising coating, in combination with said at least one fraction, at least one exogenous activator of a coagulation cascade.

17. A method of preparing a surgical mesh for repairing a hernia defect in a human or other animal subject, the method comprising:
drawing a tissue specimen from said subject;
placing said specimen into a container comprising a buoy;
centrifuging said container separating the specimen into at least two fractions that are separated by said buoy;
drawing a selected fraction from said at least two fractions from said container;
treating a surface of a biocompatible mesh; and
coating said surface with said selected fraction.

18. The method according to claim 17 wherein the said selected fraction is selected from the group consisting of platelet-rich plasma, platelet-poor plasma, cryoprecipitated plasma, concentrated platelet-poor plasma, fibrin sealant, bone marrow aspirate, concentrated bone marrow aspirate, processed lipoaspirate cells, and combinations thereof.

19. The method according to claim 17 wherein said treating said surface of said biocompatible mesh further comprises at least one of plasma etching, plasma deposition, plasma cleaning, corona discharge, chemical attack with acidic liquids, modification by exposure to gamma irradiation in the presence of a reactive gas, and combinations thereof.

20. The method according to claim 17 wherein said specimen is selected from the group consisting of whole blood, bone marrow aspirate, and combinations thereof.

21. The method according to claim 17 further comprising coating said treated surface with an optional material selected from the group consisting of antibiotics, chemotherapeutics, gene therapy agents, anti-inflammatories, clotting agents, antioxidants, growth factors, cytokines, or combinations thereof.

22. The method according to claim 17 further comprising coating said surface-treated biocompatible mesh with at least one exogenous activator of a coagulation cascade.

23. A method of repairing a hernia defect in a human or other animal subject, comprising implanting a surgical mesh made according to the process of claim 17.

24. An implant comprising:
a biocompatible surgical mesh having a treated surface; and
a selected fraction of a tissue specimen, said selected fraction coated on at least a portion of said treated surface,
wherein said selected fraction is selected from said tissue specimen that has been centrifuged in a container operable to fractionate said tissue specimen into different densities.

25. The implant according to claim 24 wherein the said biocompatible surgical mesh is selected from the group consisting essentially of plasma etching, plasma deposition, plasma cleaning, corona discharge, chemical attack with acidic liquids, modification by exposure to gamma irradiation in the presence of a reactive gas and combinations thereof.

26. The implant according to claim 24 wherein the said selected fraction is selected from the group consisting of platelet-rich plasma, platelet-poor plasma, cryoprecipitated plasma, concentrated platelet-poor plasma, fibrin sealant, bone marrow aspirate, concentrated bone marrow aspirate, processed lipoaspirate cells, and combinations thereof.

27. The implant according to claim 24 wherein the tissue specimen is selected from the group consisting of whole blood, bone marrow aspirate, and combinations thereof.

28. A method of preparing a surgical mesh for repairing a hernia defect in a human or other animal, the method comprising:
placing a tissue specimen into a container operable to separate said tissue specimen into fractions of different densities;
centrifuging said container separating the tissue specimen into at least two fractions of different densities;
drawing a selected fraction from said at least two fractions from said container; and
coating a surface-treated biocompatible mesh with said selected fraction.

29. The method according to claim 28 wherein said specimen is autologous with said subject.

30. The method according to claim 28 wherein said tissue specimen is selected from the group consisting of whole blood, bone marrow aspirate, or combinations thereof.

31. A method of repairing a hernia defect in a human or other animal subject, comprising implanting a surgical mesh made according to the process of claim 28.

32. The manufacture of a device for use in repairing a hernia defect in a human or other animal subject,
said device comprising a container comprising a buoy, said container and buoy being operable during centrifugation of said container to separate a multi-component aqueous tissue portion into two or more fractions having different densities; and
said repairing comprises drawing a tissue specimen from said subject; placing said specimen into said container; centrifuging said container so as to separate said portion into at least two fractions, drawing at least one of said fractions from said container, applying said fraction to a surface of a surface-treated biocompatible mesh; and implanting said mesh to the site of said hernia defect.

33. A hernia repair system, comprising:
a device comprising a container comprising a buoy, said container and buoy being operable during centrifugation of said container to separate a multi-component aqueous tissue portion into two or more fractions having different densities; and
a surgical process component operable to facilitate the repair of a hernial defect in a human or other animal subject using at least one of said fractions.

34. A hernial repair system according to claim 33, wherein said surgical process component comprises a surface-treated mesh, operable for coating with said one of said fractions.

35. A hernial repair system according to claim 34, wherein said surgical process component comprises surgical method instructions for said repair of a hernial defect, said instructions comprising the steps of obtaining a tissue composition comprising said one of said fractions, applying said tissue composition to a surface of a surface-treated mesh, and implanting said surface-treaded mesh to the site of said hernial defect.

36. A kit for hernia repair comprising:
a separator having a buoy; and
a surface-treated biocompatible surgical mesh.

37. The kit according to claim 36 further comprising a syringe.
